(19) United States
(12) Patent Application Publication
(10) Pub. No.: US 2023/0157981 A1
(43) Pub. Date: May 25, 2023

(54) Methods, Compositions and Devices for Treating Mild Traumatic Brain Injury, Post Traumatic Stress Disorder and Mild Traumatic Brain Injury with Post Traumatic Stress Disorder

(71) Applicant: LOBE SCIENCES LTD., Vancouver (CA)

(72) Inventors: Maghsoud DARIANI, Fanwood, NJ (US); Shlomo PIONTKOWSKI, New York, NY (US)

(21) Appl. No.: 17/916,855

(22) PCT Filed: Apr. 20, 2021
(86) PCT No.: PCT/US2021/028068

§ 371 Date: Oct. 4, 2022

Related U.S. Application Data
(60) Provisional application No. 63/012,435, filed on Apr. 20, 2020.
(60) Provisional application No. 63/016,455, filed on Apr. 28, 2020.
(60) Provisional application No. 63/059,272, filed on Jul. 31, 2020.

(51) Int. Cl. A61K 31/198; A61P 25/26; A61K 31/675; A61M 11/00; A61M 15/08; A61M 15/00

(52) U.S. Cl. CPC A61K 31/198; A61K 31/675; A61M 11/007; A61M 15/0003; A61M 15/08; A61M 15/0066; A61P 25/26

(57) ABSTRACT

Methods and compositions for treating mTBI, PTSD or mTBI with PTSD with psychedelic agents and N-acetylcysteine are provided. Nasal mist transducers for administration of one or more pharmaceutically active ingredients such as these as fine mist particles at preselected dosages and times are also provided.

This patent application claims the benefit of priority from U.S. Provisional Application Serial No. 63/059,272, filed Jul. 31, 2020, U.S. Provisional Application Serial No. 63/016,455 filed Apr. 28, 2020 and U.S. Provisional Application Serial No. 63/012,435, filed Apr. 20, 2020, the teachings of each of which are herein incorporated by reference in their entireties.

### FIELD

This disclosure relates to methods for treating or alleviating symptoms of mild traumatic brain injury (mTBI), post traumatic stress disorder (PTSD) and mTBI with PTSD via administration of a psychedelic agent in combination with N-acetylcysteine (NAC). Compositions comprising a psychedelic agent in combination with NAC for use in treating or alleviating symptoms of mTBI, PTSD and mTBI with PTSD are also disclosed. In addition, nasal mist transducers (NMT) for administration of pharmaceutical agents at preselected dosages and times is also disclosed.

### BACKGROUND

Post-traumatic stress disorder (PTSD) and traumatic brain injury (TBI) often coexist because brain injuries are often sustained in traumatic experiences (Bryant, R. Dialogues Clin Neurosci. 2011 3:251-262).

TBI involves damage to the brain from an external force. Brain injuries can involve contusion, brain laceration, intracranial hematoma, contrecoup injury, shearing of nerve fibers, intracranial hypertension, hypoxia, anemia, metabolic anomalies, hydrocephalus, and subarachnoid hemorrhage (Bryant, R. Dialogues Clin Neurosci. 2011 3:251-262). Severity of TBI is typically described in terms of mild or moderate/severe with mild traumatic brain injury (mTBI) usually being defined as: (i) an external injury to the brain; (ii) confusion, disorientation, or loss of consciousness for 30 minutes or less; (iii) Glasgow Coma Scale score of 13 to 15; and (iv) post-traumatic amnesia for less than 24 hours (American Congress of Rehabilitation Medicine. Definition of mild traumatic brain injury. J Head Trauma Rehab. 1993 8:86-87; Carroll et al. J Rehab Med. 2004 36:113-125; Ruff et al. Arch Clin Neuropsychol. 2009 24:3-10) .

PTSD reactions can be immediate or longer-term and are distinguished diagnostically because acute stress reactions are frequent, but often transient, as compared to the less common persistent PTSD responses. In terms of the persistent responses, PTSD is described in the American Psychiatric Association’s DSM-IV as an anxiety disorder that comprises five major criteria (American Psychiatric Association. Diagnostic and Statistical Manual of Mental Disorders. 4th ed. Washington, DC: American Psychiatric Association 1994). First, one must have been exposed to or witness an event that is threatening to safety, and one must respond to this event with fear, horror, or helplessness. Second, one must report a re-experiencing symptom, which may include intrusive memories, nightmares, a sense of reliving the trauma, or psychological or physiological distress when reminded of the trauma. Third, there need to be at least three avoidance symptoms, which can include active avoidance of thoughts, feelings, or reminders of the trauma, inability to recall some aspect of the trauma, withdrawal from others, or emotional numbing. Fourth, one must suffer marked arousal, which can include insomnia, irritability, difficulty concentrating, hypervigilence, or heightened startle response. Finally, these symptoms must cause marked impairment to one’s functioning, and can only be diagnosed when they are present at least 1 month after the trauma.

It was previously argued that PTSD could not develop following TBI because the impaired consciousness at the time of trauma precluded encoding of the traumatic experience, and this prevented trauma memories that are necessary for PTSD development (Sbordone R.J. & Liter J.C. Brain Inj. 1995 9:405-412; Price K.P. Law J. 1994 43:113-120). More recently, however, evidence has accumulated that PTSD can develop following mild TBI (Bryant R.A. & Harvey A.G. Am J Psychiatry 1998 155:625-629; Middelboe et al. Eur Psychiatry. 1992 7:183-189; Ohry et al. Brain Inj. 1996 10:687-695; Hickling et al. Brain Inj. 1998 12:265-274; Castro C.A. & Gaylord K.M. J Trauma-Inj Infect Crit Care. 2008 64:S205-S206; Greenspan et al. Brain Inj. 2006 20:733-742; Harvey A.G. & Bryant R.A. Am J Psychiatry. 2000 157:626-628; Hoge et al. N Engl J Med. 2008 358:453-463; Levin et al. J Clin Exp Neuropsychol. 2001 23:754-769.

Several models have been set forth to explain how PTSD can develop following TBI including fear conditioning, memory reconstruction and postamnesia resolution (Bryant, R. Dialogues Clin Neurosci. 2011 3:251-262).

The definitions of postconcussive syndrome (PCS) can vary, but generally overlap somewhat with symptoms of PTSD. For example, the International Classification of Diseases (ICD-10) stipulates that PCS is defined by headaches, dizziness, general malaise, fatigue, noise intolerance, irritability, emotional lability, depression, or anxiety, concentration or memory difficulty, sleep disturbance, reduced tolerance to alcohol, and a preoccupation with these symptoms and fear of permanent brain damage (World Health Organization. The ICD-10 Classification of Mental and Behavioural Disorders: Clinical Descriptions and Diagnostic Guidelines. 1995 ed. Geneva, Switzerland: World Health Organization. 1995). The Appendix of the DSM-IV describes PCS as fatigue, sleep disturbance, headaches, dizziness, irritability, anxiety or depression, changes in personality, and apathy (American Psychiatric Association. Diagnostic and Statistical Manual of Mental Disorders. 4th ed. Washington, DC: American Psychiatric Association. 1994). These descriptions clearly overlap with common symptoms of post-traumatic stress.

N-acetylcysteine (also known as acetylcysteine or N-acetyl-L-cysteine or NAC) is primarily used as a mucolytic agent and in the management of acetaminophen poisoning. It is a derivative of cysteine with an acetyl group attached to the amino group of cysteine. NAC is essentially a prodrug that is converted to cysteine (in the intestine by the enzyme aminoacylase 1) and absorbed in the intestine into the blood stream. Cysteine is a key constituent to glutathione, which is an antioxidant capable of preventing damage to important cellular components caused by reactive oxygen species such as free radicals, peroxides and lipid peroxides. Hence, administration of NAC replenishes glutathione levels in the body, which can help mitigate symptoms for a variety of diseases exacerbated by reactive oxygen species (ROS). For instance, NAC is commonly used in individuals with renal impairment to prevent the precipitation of acute renal failure. NAC has also been shown to have efficacy in treating mild to moderate traumatic brain injury including ischemic brain injury, particularly in reducing neuronal losses, and also reducing cognitive and neurological symptoms when administered promptly after injury. In addition, NAC has been shown to have anti-inflammatory activities by inhibiting expression of proinflammatory cytokines. NAC is also being successfully used to treat a variety of neuropsychiatric and neurodegenerative disorders including cocaine, cannabis, and smoking addictions, Alzheimer’s and Parkinson’s diseases, autism, compulsive and grooming disorders, schizophrenia, depression, and bipolar disorder.

Psychedelics are a subset of hallucinogenic drugs whose primary effect is to trigger non-ordinary states of consciousness (known as psychedelic experiences or “trips”) via serotonin 2A receptor agonism. This causes specific psychological, visual and auditory changes, and often a substantially altered state of consciousness. Psychedelics with the largest scientific and cultural influence include mescaline, lysergic acid diethylamide (LSD), psilocybin, and N,N-Dimethyltryptamine (DMT). Studies show that psychedelics are physiologically safe and do not lead to addiction (Le Dain, G (1971). The Non-medical Use of Drugs: Interim Report of the Canadian Government’s Commission of Inquiry. p. 1061 Lüscher, C & Ungless, M.A. PLOS Medicine. 2006 3 (11): e4370). Although further research is needed, existing results are showing that psychedelics may be useful for treating certain forms of psychopathology (Garcia-Romeu et al. Experimental and Clinical Psychopharmacology. 2016 24 (4): 229-268; Friedman, H. The Humanistic Psychologist. 2006 34 (1): 39-58; Tupper et al. CMAJ: Canadian Medical Association Journal. 2015 187 (14): 1054-1059).

For example, active ingredients in Psilocybe cubensis, psilocybin and/or psilocycin create a sympathetic arousal state characterized by euphoria, visual and mental hallucinations, changes in perception, a distorted sense of time, spiritual experiences, giddiness, joy, open and closed eye visuals common at medium to high doses, along with synesthesia (e.g. hearing colors and seeing sounds). The mind-altering effects of psilocybin typically last from two to six hours. Adverse reactions include nausea, disorientation, lethargy and depression and panic attacks with about a third of users reporting feelings of anxiety or paranoia. Additional side effects include tachycardia, dilated pupils, restlessness or arousal, increased body temperature, headache, sweating and chills.

These effects are the result of psilocybin’s rapid metabolism to psilocin, which then activates or partially activates several serotonin receptors including 5-HT2A, 5-HT2B and 5-HT2C in the brain. It is widely accepted that the hallucinogenic effects are generated primarily by agonist activity at the serotonin 5-HT2A receptor. Psilocin further binds with low affinity to 5-HT1 receptors, including 5-HT1A and 5-HT1D. In addition, psilocin indirectly increases the concentration of the neurotransmitter dopamine in the basal ganglia.

The benefits of psilocybin in the treatment of depression, anxiety and other disorders were first suggested in the 1960s when psilocybin was marketed in many countries, including the United States, under the trade name Indocybin® by the Swiss pharmaceutical company, Sandoz. Indocybin® provided a shorter acting alternative to lysergic acid diethylamide (LSD) which has a similar primary pharmacological mechanism of action, now known to be agonist or partial agonist effects at the 5-HT2A receptor (Nichols, 2016). While Indocybin® was used safely as an adjunct to psychotherapy, eventually the societal backlash in the US and other countries in the 1960s (Matsushima et al., 2009) led to a ban on marketing and possession of “hallucinogenic” drugs in the US in 1965, and led Sandoz to discontinue manufacturing and marketing of Indocybin® in 1966 (Belouin and Henningfield, 2018; Bonson, 2018; Novak, 1997). The 1970 placement of psilocybin, LSD, and other “hallucinogens” in Schedule I of the CSA did not reflect an absence of therapeutic benefit, although the scientific evidence at the time was mixed.

Published U.S. Application Nos. 2018/0221396 and 2019/0142851 disclose methods and compositions comprising a psilocybin derivative selected from [3,2-dimethylaminoethyl)-lH-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, [3-(2-trimethylaminoethyl)-1H-indol-4-yl] dihydrogen phosphate and 4-hydroxy-N,N,N-trimethyltryptamine for regulating serotonin alone or in combination with a cannabinoid and/or terpene in purposely engineered with unnaturally occurring molar ratios.

Published U.S. Pat. Application No. 2019/0105313 discloses compositions comprising fungal extracts and their active ingredients including species of mushrooms and mycelia containing psilocybin and psilocin, combined with ernicines and hericenones or fungal extracts containing those active ingredients with the addition of nicotinic acid.

Lysergic acid diethylamide (LSD) was studied from the 1950s to the 1970s to evaluate behavioral and personality changes, as well as remission of psychiatric symptoms in various disorders. LSD was used in the treatment of anxiety, depression, psychosomatic diseases and addiction. In more recent studies, LSD was administered to 567 patients in a dose ranging from 20 to 800 mcg and positive results were observed, thus revealing the therapeutic potential of LSD to reduce psychiatric symptomatology, mainly in alcoholism. See frontiersin with the extension .org/articles/10.3389/fpsyt. 2019.00943/full of the world wide web.

Dimethyltryptamine (DMT) is an intense naturally-occurring psychedelic that’s also found endogenously in the human body.

Mescaline is a psychedelic hallucinogen obtained from the small, spineless cactus Peyote (Lophophora williamsi), the San Pedro cactus, Peruvian torch cactus, and other mescaline-containing cacti. It is also found in certain members of the Fabaceae (bean family) and can be produced synthetically. Mescaline has a wide array of suggested medical usage, including treatment of alcoholism and depression, due to these disorders having links to serotonin deficiencies.

Administration of a psychedelic agent and NAC is expected to be useful in alleviating symptoms associated with mTBI, PTSD and mTBI with PTSD.

### SUMMARY

An aspect of the present invention relates to a method for alleviating one or more symptoms of mild traumatic brain injury (mTBI), post-traumatic stress disorder (PTSD) and/or mTBI with PTSD. The method comprises administering to an individual suffering from mTBI, PTSD or mTBI with PTSD a psychedelic agent and N-acetylcysteine (NAC).

Another aspect of the present invention relates to a method for alleviating one or more symptoms of mTBI, PTSD) and/or mTBI with PTSD which comprises administering to an individual suffering from mTBI, PTSD or mTBI with PTSD a psychedelic agent and NAC in combination with memory-odor imprint pairing.

Another aspect of the present invention relates to pharmaceutical formulations and kits thereof comprising a psychedelic agent and NAC for use in alleviating one or more symptoms of mTBI, PTSD and/or mTBI with PTSD.

Another aspect of the present invention relates to kits comprising a psychedelic agent, NAC and an odor for memory odor imprint pairing for use in alleviating one or more symptoms of mTBI, PTSD and/or mTBI with PTSD.

In one nonlimiting embodiment, one or more of the psychedelic agent and NAC are administered intranasally to alleviate one or more symptoms of mTBI, PTSD and/or mTBI with PTSD.

Another aspect of the present invention relates to devices, referred to herein as nasal mist transducers (NMT), for administration of one or more pharmaceutical ingredients as fine mist particles at preselected dosages and times.

In one nonlimiting embodiment, the device delivers the agents sequentially as distinct dosages.

In one nonlimiting embodiment, the device is used to administer a psychedelic agent and/or NAC.

In one nonlimiting embodiment, the NMT delivers the one or more pharmaceutical ingredients deep into the nasal cavity vestibule at close proximity to the olfactory bulb where the deep and superficial veins drain directly to the circulatory system of the brain.

Another aspect of the present invention relates to a method for administering one or more pharmaceutical ingredients to the circulatory system of the brain via administration of the one or more pharmaceutical ingredients via the NMT.

Yet another aspect of the present invention relates to a method for treating or alleviating symptoms associated with mTBI, PTSD and/or mTBI with PTSD. The method comprises administering to a subject suffering from mTBI, PTSD or mTBI with PTSD a psychedelic agent and NAC via the NMT.

### BRIEF DESCRIPTION OF THE FIGURES

FIG. 1 is a diagram depicting the anatomical positioning of a nasal mist transducer (NMT) of the present invention.

FIG. 2 is a diagram showing elements of a nonlimiting embodiment of an NMT of the present invention.

FIG. 3 shows a closer view of a medication container useful in the NMT of the present invention.

FIG. 4 shows a closer view of a nonlimiting embodiment of a mist generator useful in the NMT of the present invention.

FIG. 5 shows a closer view of a nonlimiting embodiment of a syringe loading apparatus useful in the NMT of the present invention.

FIG. 6 shows a closer view of a nonlimiting embodiment of a hydraulic propulsion mechanism useful in the NMT of the present invention.

### DETAILED DESCRIPTION

The present invention provides methods and compositions for alleviating one or more symptoms of mild traumatic brain injury (mTBI), post-traumatic stress disorder (PTSD) and/or mTBI with PTSD.

The methods and compositions involve administration of a psychedelic agent in combination with N-acetylcysteine (NAC) .

By “psychedelic agent” as used herein, it is meant a drug from the subset of hallucinogenic drugs whose primary effect is to trigger non-ordinary states of consciousness (known as psychedelic experiences or “trips”) via serotonin 5HT2A receptor agonism. Nonlimiting examples include mescaline, lysergic acid diethylamide (LSD), psilocybin or a psilocybin-derived agent, and N,N-Dimethyltryptamine (DMT).

In one nonlimiting embodiment, the psychedelic agent is psilocybin or a psilocybin-derived agent.

Psilocybin is rapidly metabolized to psilocin, which then acts on serotonin receptors in the brain. It partially activates several serotonin receptors including 5-HT2A, 5-HT2B and 5-HT2C in the brain. It is widely accepted that the hallucinogenic effects are generated primarily by agonist activity at the serotonin 5-HT2A receptor. Psilocin further binds with low affinity to 5-HT1 receptors, including 5-HT1A and 5-HT1D. In addition, psilocin indirectly increases the concentration of the neurotransmitter dopamine in the basal ganglia. Finally, psilocin is degraded by the enzyme monoamine oxidase in the liver, lungs and gut.

Nonlimiting examples of psilocibe-derived agents which can be used in the present invention include psilocybin and psilocin as well as 3,2-dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, [3-(2-trimethylaminoethyl)-1H-indol-4-yl] dihydrogen phosphate and 4-hydroxy-N,N,N-trimethyltryptamine] .

When administered intranasally, via for example a nasal mist transducer as disclosed herein which delivers the agent almost directly to the brain, administration of psilocin may be more effective.

N-acetylcysteine (NAC) is a potent antioxidant, via increasing the levels of glutathione levels in the body, which can help protect brain cells from reactive oxygen species and trauma to the head. N-acetylcysteine has been shown to have efficacy in treating mild to moderate traumatic brain injury including ischemic brain injury, particularly in reducing neuronal losses, and also reducing cognitive and neurological symptoms when administered promptly after injury.

As used herein, by alleviating one or more symptoms of mTBI, PTSD and/or mTBI with PTSD is it meant to decrease severity of one or more of intrusive memories, nightmares, a sense of reliving the trauma, or psychological or physiological distress when reminded of the trauma, active avoidance of thoughts, feelings, or reminders of the trauma, inability to recall some aspect of the trauma, withdrawal from others, or emotional numbing, insomnia, irritability, difficulty concentrating, hypervigilence, or heightened startle response. The inventors believe that the studies disclosed herein will demonstrate that the combination therapy of psychedelic agent and NAC will be more effective in alleviating one or more symptoms of mTBI, PTSD and/or mTBI with PTSD than either agent individually. Preferred combination therapies in accordance with this invention are synergistic, meaning better than additive in their efficacy in alleviating one or more symptoms of mTBI, PTSD and/or mTBI with PTSD.

In one nonlimiting embodiment, the psychedelic agent and NAC are administered in combination immediately following the mTBI or within 12 to 24 hours of the mTBI. In one nonlimiting embodiment, the psychedelic agent and NAC are administered in combination upon the onset of symptoms of PTSD. In one nonlimiting embodiment, the psychedelic agent and NAC are administered after a traumatic event typically leading to PTSD. As will be understood by the skilled artisan upon reading this disclosure, dosages can be determined by the attending physician, according to the extent of the injury to be treated, method of administration, patient’s age, weight, contraindications and the like.

As used herein, by “in combination” it is meant to include coadministration of the psychedelic agent and NAC, sequential administration of the psychedelic agent followed by NAC, or sequential administration of NAC followed by the psychedelic agent.

In one nonlimiting embodiment, NAC is administered within 12 hours of the traumatic brain injury, or alternatively with 6 hours of the traumatic brain injury, or alternatively within 3 hours of the traumatic brain injury. In these embodiments, NAC may be administered as a single dose or as multiple doses.

In one nonlimiting embodiment, multiple doses of NAC are administered over a 72 hour period following the traumatic brain injury.

In one nonlimiting embodiment, NAC is administered daily or every two days until symptoms of the traumatic brain injury are alleviated.

In one nonlimiting embodiment, NAC is administered upon onset of symptoms of PTSD.

In one nonlimiting embodiment, NAC is administered within 3 to 24 hours of a traumatic event which typically results in PTSD. In this embodiment, NAC may be administered as a single dose or as multiple doses.

In one nonlimiting embodiment, multiple doses of NAC are administered over a 72 hour period following the traumatic event.

NAC may be administered by any route providing for delivery of effective amounts to the brain. Examples of routes of administration include, but are in no way limited to, intravenous, intranasal, oral, topical, transdermal or via inhalation.

Doses of NAC which have been administered safely for various conditions in humans range from 70 mg up to 6 grams per day. See webmd with the extension com/vitamins/ai/ ingredientmono-1018/n-acetyl-cysteine-nac of the world wide web. As will be understood by the skilled artisan upon reading this disclosure, similar dosing regimens to those already used for NAC as well as alternative dosing regimens determined to be clinically relevant may be used.

Doses and routes for administration for psychedelic agents will vary depending upon the psychedelic agent selected for administration. Selection may be based upon similar dosing regimens known in the art to be safe while exhibiting pharmacological activity. As nonlimiting examples, LSD has been administered in doses ranging from 20 to 800 micrograms; DMT has been administered in doses ranging from 10-60 milligrams both orally and via inhalation; dosages is 200-400 milligrams of mescaline sulfate and dosages of 178-356 milligrams of mescaline hydrochloride have been administered; and therapeutic ranges of 20 to 30 mg/70 kg of psilocybin have been disclosed. As will be understood by the skilled artisan upon reading this disclosure, similar dosing regimens to those already used for these psychedelic agents as well as alternative dosing regimens determined to be clinically relevant may be used.

In addition, psychedelic microdosing, a practice of using sub-threshold doses (microdoses) of serotonergic psychedelic drugs may be used.

The psychedelic agent can be administered before, simultaneouslyor after administration of the NAC.

In one nonlimiting embodiment, the psychedelic agent and NAC are coadministered in a solid dosage formulation.

In one nonlimiting embodiment, an encapsulation technique is used to enclose various concentrations of the psychedelic agent and NAC in a relatively stable shell known as a capsule, allowing them to, for example, be taken orally. In one nonlimiting embodiment, the formulation of the present invention comprises a hard-shelled capsule containing dry, powdered ingredients, miniature pellets made by processes such as extrusion and spheronization or mini tablets. The hard-shelled capsules are typically made in two halves: a smaller-diameter body that is filled and then sealed using a larger-diameter cap. The capsule itself is typically made from aqueous solutions of gelling agents, such as animal protein (mainly gelatin) or plant polysaccharides or their derivatives (such as carrageenans and modified forms of starch and cellulose). Other ingredients can be added to the gelling agent solution including plasticizers such as glycerin or sorbitol to decrease the capsule’s hardness, coloring agents, preservatives, disintegrants, lubricants and surface treatment.

In one nonlimiting embodiment, the psychedelic agent and NAC are coadministered in a nasal spray formulation.

In one nonlimiting embodiment, the psychedelic agent and NAC are administered sequentially in a nasal spray or mist transducer (NMT) programmed time release administration.

In one nonlimiting embodiment, the psychedelic agent and NAC are coadministered in a nasal spray where therapeutically active amounts of each are dissolved or suspended in solutions or mixtures of excipients (e.g., preservatives, viscosity modifiers, emulsifiers, buffering agents) in nonpressurized dispensers that deliver a spray containing a metered dose of each ingredient.

In one nonlimiting embodiment, coadministration of the psychedelic agent and NAC enables pathological memory eradication for treatment of mTBI, PTSD and/or mTBI with PTSD.

In one nonlimiting embodiment, coadministration of the psychedelic agent and NAC is expected to prevent or inhibit pathological conversion of short term memory (STM) to pathological long term memory (LTM) and promote disengagement of pathological LTM by a chemical agonist/antagonist shock similar to insulin and/or electric shock therapy. Such formulations are expected to be useful in treating disorders related to pathological LTM such as mTBI, PTSD and mTBI with PTSD.

In one nonlimiting embodiment, the psychedelic agent and NAC are administered in combination with memory-odor imprint pairing. In one nonlimiting embodiment, the odor is administered to the nasal vestibule via an NMT. It is expected that exposure to an odor immediately or shortly after a trauma or electively any time thereafter during memory of the trauma, followed by multiple odor-memory pairing sessions thereafter, will elicit a Pavlovian reaction to the odor.

Memory pairing and avoidance of memory recall was demonstrated by Pavlov in his well-known dog experiment. Pavlov’s dogs initially salivated at the sight and/or smell of food. When paired (tagged) with the sound of a bell, the dog eventually salivated only at the sound of the bell without sight or smell of food. Eventually the dogs did not anticipate food unless the bell rang; in essence they forgot about the food because there were no bell stimuli, they had no memory of the food.

Similarly, classical conditioning occurs in subjects when a conditioned stimulus (real, for example the smell of food, or imaginary, for example imagining a lemon or remembering a deceased loved one which promotes a conditioned response such as salivation or tears) is paired with an unconditioned stimulus (for example a smell or sound) which does not promote the conditioned response. After tagging or pairing is repeated sufficient times, a subject will exhibit the conditioned response to the unconditioned stimulus when it is presented alone (ex: bell ringing).

In one embodiment of the present invention, classical conditioning is used to pair pathologic memories, emotions and/or thoughts of a trauma associated with PTSD in a subject to an unconditioned stimulus of an odor, such as, but in no way limited to, lavender. This allows for subsequent negation of the distinct olfactory sensor for this odor in the subject either chemically with a drug such as lidocaine or by surgically removing or extinguishing an olfactory bulge explicit for the odor. Elimination of the smell suppresses or eradicates the Pavlovian paired pathologic emotion(s)/ memories(s)/thought(s) by impeding memory and emotion resurfacing from the subconscious LTM pool and becoming a current STM. Should resurfacing occur, administration of the psychedelic agent and NAC, preferably via NMT in this combination therapy, will repress it back into the LTM pool or the subconscious.

Also provided in the present invention are devices, referred to herein as a nasal mist transducers (NMTs), for administration of one or more pharmaceutical agents at preselected dosages and times as fine mists deep into the nasal cavity vestibule at close proximity to the olfactory bulb where the deep and superficial veins drain directly to the circulatory system of the brain. Such delivery provides for fast absorption with almost instantaneous drug penetration of the blood-brain barrier. Thus, NMTs of the present invention provide for superior access of active pharmaceutical ingredients to the brain and its constituents thereby resulting in enhanced clinical and physiological effects as compared to presently available nasal and non-nasal drugs dispensing devices and formulation.

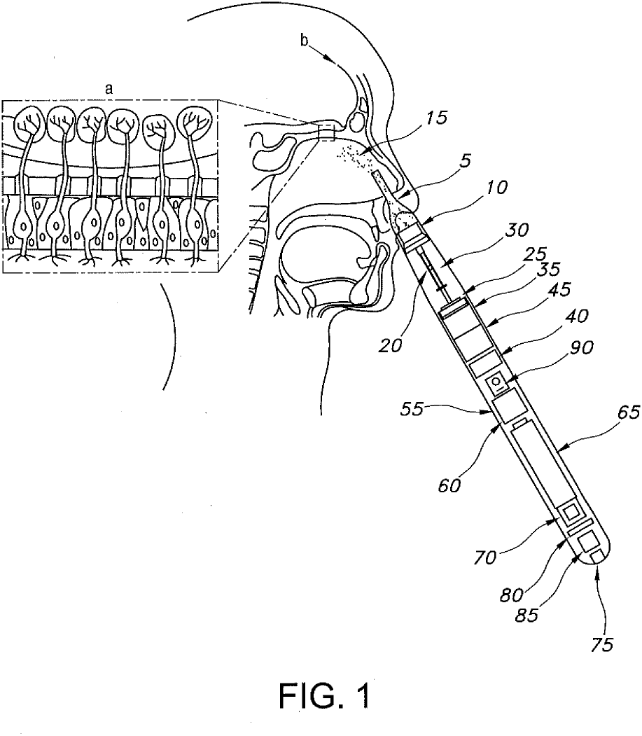

FIG. 1 is a diagram depicting the anatomical positioning of a nonlimiting embodiment of a Nasal Mist Transducer (NMT) of the present invention. As shown, the NMT is situated deep into the nasal vestibule at close proximity to the olfactory bulb a where the blood-brain barrier b is easily circumvented by virtue of the fine mist generated by the NMT and the anatomical uniqueness of the nasal mucosa there, whereby superficial and deep veins drain directly into brain’s circulation, as opposed to draining toward the right heart chamber as most other veins do. This provides for a faster drug to brain introduction and enables drug(s) dosage control and resulting physiological effect.

In simplest form, the NMTs of the present invention comprise a nasal funnel capable of fitting into a vestibular anatomy of a human a mist generator with a top and bottom which produces a fine mist at the top which is propelled toward the nasal funnel, a syringe loading apparatus capable of holding one or more micro syringes attached at the bottom of the mist generator, and a means for applying pressure to a plunger of a microsyringe loaded into the syringe loading apparatus.

By “fine mist” as used herein, it is meant a plurality of droplets produced from the content of a microsyringe ranging in size from about 30 to about 100 microns.

A nonlimiting embodiment of an NMT of the present invention is depicted in FIGS. 1-6. As will be understood by the skilled artisan upon reading this disclosure, however, alternative components having similar function resulting in the device still delivering a fine mist to the nasal vestibule at close proximity to the olfactory bulb where the blood-brain barrier is easily circumvented by virtue of the fine mist generated by the NMT and the anatomical uniqueness of the nasal mucosa there can be routinely substituted and are encompassed within the scope of this invention.

A nonlimiting embodiment of an NMT of the present invention is depicted in FIGS. 1-6. As shown therein, the NMT comprises a nasal funnel 5 which is soft and accommodates each individual’s distinct vestibular anatomy, thus making it comfortable. The NMT further comprises a mist generator 10 which produces a fine mist 15 and propels it toward the nasal funnel 5.

In this nonlimiting embodiment depicted in FIGS. 1-6, pre-loaded micro syringes 20 of pharmaceutical agents such as a psychedelic agent and NAC are stationed onto a syringe loading apparatus 25 constrained within a medication container 30 of the NMT. A rotating straining disc 35 is activated by circuit board chip 40 pre-programed with a selected dosing algorithm. The rotating straining disc 35 rotates and allows explicit measured hydraulic pressure generated by the propulsion mechanism 45 on the one and only exposed micro syringe plunger 50. This dictates distinct pharmaceutical dosage induction and timing for desired physiological and clinical outcomes. A digital display 55 displays the programed algorithm and allows for NMT algorithm, time and alarm setup.

Alternatively, motion of the microsyringes can be controlled via a multiaxis motion control system such as, but not limited to, the TinyG (see https with the extension synthetos.myshopify.com/products/tinyg of the world wide web) .

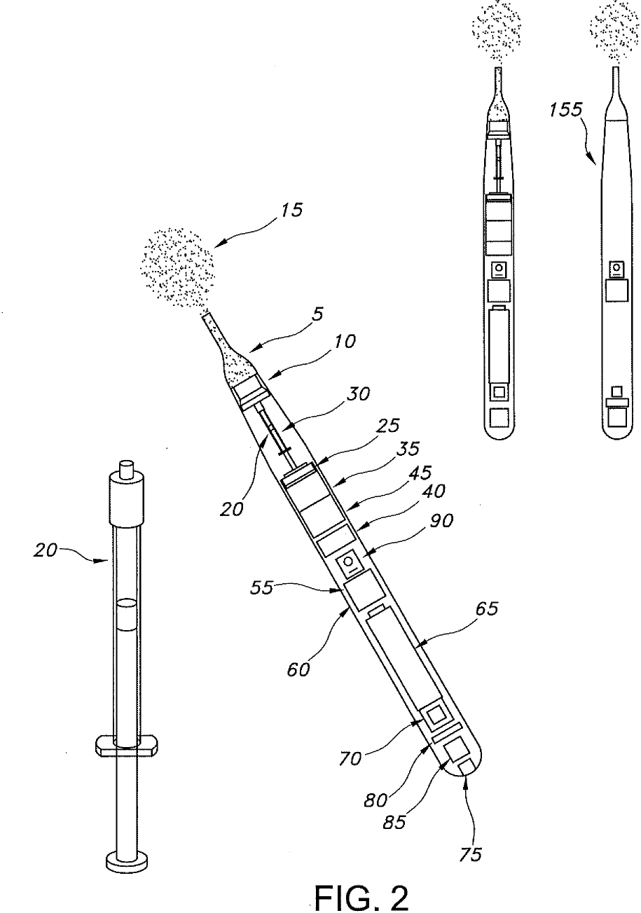

The device further comprises a power source. In one nonlimiting embodiment, as depicted in FIGS. 1 and 2, the power source comprises a transducer 60 powered by a rechargeable battery 65 with power level display 70 charged via a micro USB 75. In some embodiments, the device further comprises a mini speaker 80 which provides for sounding an alarm and/or vocalized programming instructions. Such instructions can also be embedded in a clearly visible bar code 85 decipherable by a mobile phone application. The NMT can be self-activated via on/off switch 90, or by a medical professional or other trained personnel such as a health coach.

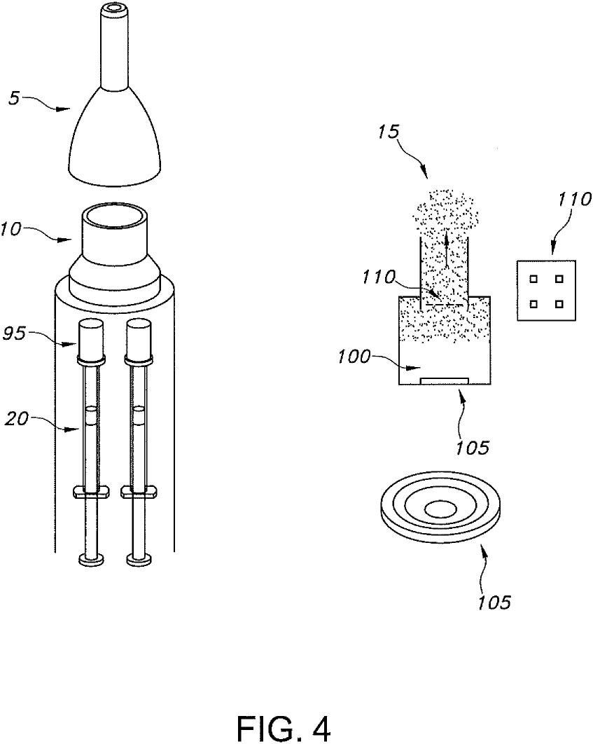

FIG. 4 shows a closer view of a nonlimiting embodiment of a mist generator 10 with a unique structure and mechanism for use in the NMT devices. Pharmaceutical ingredients navigate from the syringe cap(s) 95 and aggregate in a reservoir 100 equipped with a piezoelectric transducer 105. In one nonlimiting embodiment, the piezoelectric transducer is a thin crystal piezoelectric transducer. The piezoelectric transducer 105 converts electrical energy into mechanical energy and generates ultrasonic waves which agitate any pharmaceutical ingredient containing liquid in the reservoir 100 to form fine liquid microwaves which then break into airborne microparticles comprising the pharmaceutical ingredient which traverse a micromembrane 110, thus producing an extremely fine mist 15 of pharmaceutical ingredient which is propelled toward the nasal funnel 5 and subsequently to the nasal vestibule of a subject.

As will be understood by the skilled artisan upon reading this disclosure, however, alternative mist generators such as, but not limited to, atomizers can be used.

NMT devices of the present invention may further comprise one or more preloaded microsyringes 20 comprising selected dosages of one or more pharmaceutical ingredients positioned onto the syringe loading apparatus 25. In one nonlimiting embodiment, the NMT device comprises a first preloaded micro syringe comprising a psychedelic agent and a second preloaded micro syringe comprising NAC.

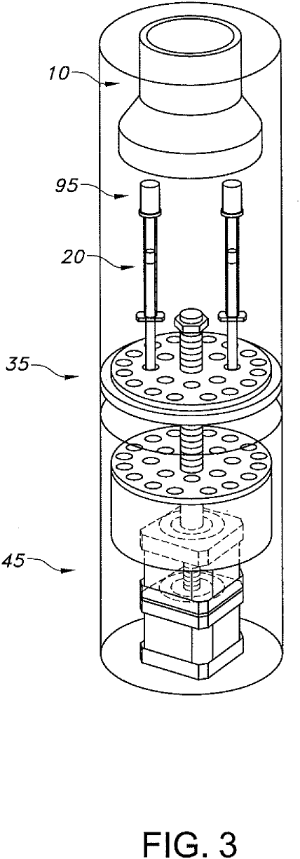

FIG. 3 shows a closer view of a nonlimiting embodiment of a medication container 30 useful in the NMT of the present invention. In this nonlimiting embodiment, the microsyringes 20 are constrained within a medication container 30 and a rotating straining disc 35 which is activated by a circuit board chip 40 pre-programed with a dosing algorithm. The rotating straining disc 35 rotates to expose a micro syringe to the mist generator 10 and allows explicit measured hydraulic pressure generated by a propulsion mechanism 45 on the exposed micro syringe plunger 50. This dictates distinct pharmaceutical dosage induction and timing for desired physiological and clinical outcomes. The medication container has an opening for insertion and extraction of any pre-loaded microsyringe(s) 20. Each micro syringe 20 has a top 115 from which ingredients are expelled and a bottom 120 in which a plunger 50 is inserted. The plunger 50 is secured onto the syringe loading apparatus 25 adjacent to the hydraulic propulsion mechanism 45.

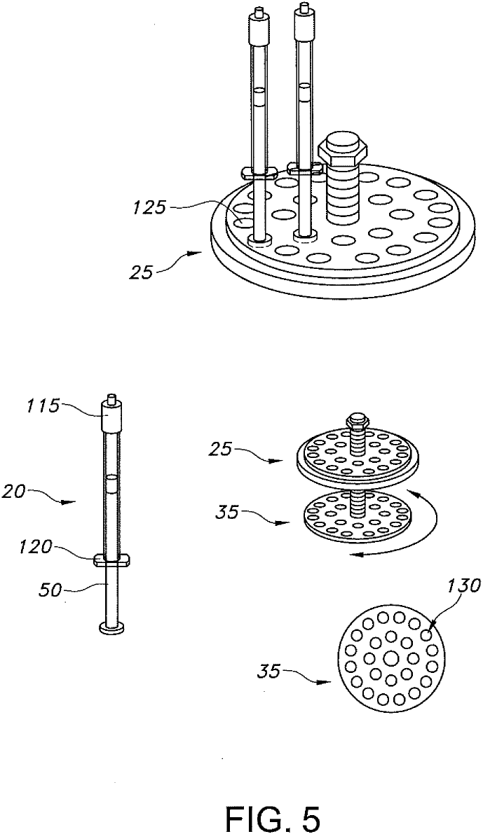

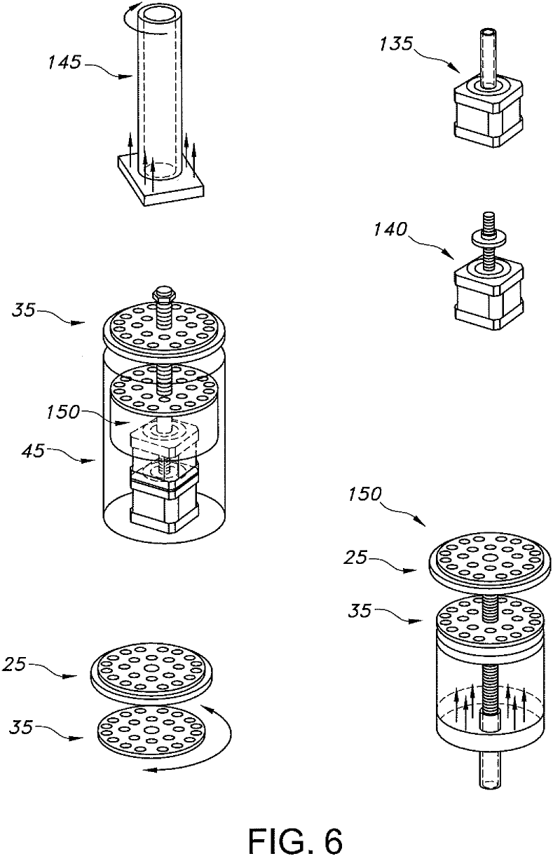

A closer view of a nonlimiting embodiment of a syringe loading apparatus 25 useful in an NMT of the present invention is depicted in FIG. 5. As shown therein, the plunger 50 of each micro syringe 20 loaded with a selected pharmaceutical ingredient is secured into separate stationary designated ports 125 of the syringe loading apparatus 25. A rotating straining disc 35 with strategically placed perforations 130 then rotates clockwise or counterclockwise in such a way as to allow for an individual micro syringe loaded in the apparatus to be exposed to a measured hydraulic pressure generated by the hydraulic propulsion mechanism 45, thereby allowing for drug(s) dosage(s) specificity injection into the mist generator 10 as per a programmed algorithm. FIG. 6 shows a closer view of a nonlimiting embodiment of a hydraulic propulsion mechanism 45 useful in the NMT of the present invention as means for applying pressure to a plunger of a microsyringe loaded into the syringe loading apparatus. This system does not involve gas canisters presently used by commercial nasal sprays and therefore does not violate any environmental restrictions imposed on fluorocarbons since 2003. Its propulsion mechanism utilizes two (2) microelectric motors 135 and 140, which exhibit a solid axle within a hollow axle 145. Engine 135 activates the rotating straining disc 35, while engine 140 elicits controlled pressure within a micro oil drum 150 which is transmitted onto a selective port 125 in the syringe(s) loading apparatus 25. The motors 135 and 140, operate independently. The rotating straining disc 35 can rotate clockwise or counterclockwise thereby allowing explicit oil leakage into only one selectively exposed syringe anchoring ports 125 which exerts hydraulic pressure on the exposed syringe plunger 50 to elicit a chosen quantity and therefore potency of drug to be dispensed through the syringe tip and onto the transducer’s mist generator 10.

As will be understood by the skilled artisan upon reading this disclosure, alternative means for applying pressure to the plunger such as, but not limited to, a linear actuator, can be used.

In some embodiments, as depicted in FIG. 2, the NMT further comprises a cover 150 which may be translucent or solid.

In one nonlimiting embodiment, the NMT is used to administer one or more pharmaceutical ingredients to the circulatory system of the brain.

In one nonlimiting embodiment, the NMT is used to administer a psychedelic agent and NAC at preselected dosages and times for the treatment or alleviation of symptoms of mTBI, PTSD and/or mTBT with PTSD.

The following nonlimiting examples are provided to further illustrate the present invention.

### EXAMPLES

### Animal Model for mTBI and PTSD

Small animal models, in particular mice and rats, are essential in the study of mTBI and PTSD. See Schoner J et al. J Cell Mol Med. 2017 (10):2248-2256; Prater et al. Neuropsychopharmacology. 2017 42(8):1706-1714; and Perez-Garcia et al. Neuropharmacology. 2019 145(Pt B):220-229. These animal models allow investigators to study the functional impact of both insults and to examine the anatomic pathologic correlates. Moreover, these animals allow investigators to include enough animals to overcome the natural heterogeneity of both disorders (mTBI and PTSD).

Rats provide an excellent model to study changes in behavior since rats are amenable to the training necessary to display the characteristic responses of PTSD (which involves changes in behavior of a previous trained and reliable model behavior). Further, rats are hardier and a better model for the dual insult of mTBI and PTSD.

### Materials and Methods Experimental Design

Five main exposure groups are examined as follows: 1) No exposure, 2) Sham fluid percussion (surgical prep but no fluid percussion injury) plus PTSD trigger, 3) FP plus PTSD trigger, 4) Blast plus PTSD trigger, 5) Repeated Blast (known to be a PTSD trigger)alone. Each exposure is detailed below. In each of the 5 groups there will be four dosing paradigms as follows A) Vehicle alone, NAC alone, psychedelic alone and D) NAC plus psychedelic. Preferred is that 12-15 rats are examined per group. However, as will be understood by the skilled artisan upon reading this studies, positive results from smaller groups are also demonstrative of efficacy. Comparisons are made between the performance of the rats within each group on each test using stated statistical methods to assess group mean differences (ANOVA, etc.)

### Methodology in Detail

Gavage - A powder comprising a combination of NAC and the psychedelic agent psilocybin, hereinafter PS, is solubilized in sterile water. The aqueous solution is then given orally by gavage to the animals once daily for seven days beginning within one hour of exposure and continuing for six more daily doses. Doses administered are as follows:

8 mg/mL NAC and 0.5 mg/mL of PS. 1 ml of each per gavage (equivalent to 2.5 mg/kg total of PS and 20 mg/kg total of NAC per gavage per animal).

Production of mTBI - Two mTBI models are utilized for this experiment: a fluid percussion model (mild - moderate mTBI) and a blast model (mild mTBI)

### Fluid Percussion Model

Day 1: For surgical preparation for the injury cap, isoflurane anesthesia is maintained via nose cone and the injury cap is placed on the exposed dura as follows. The rat’s head is shaved and swabbed with clorohexadine solution. The rat is then placed in a stereotaxic frame and the scalp surgically incised. A parasagittal craniotomy (4.8 mm) using a trephine is performed at 3.8 mm posterior to bregma and 2.5 mm lateral to the midline. A sterile plastic injury tube (the plastic connector of a sterile needle cut 1 cm in length and trimmed to fill the craniotomy perfectly) is next placed over the exposed dura and bonded by crynoacrylic adhesive to the skull. Dental acrylic is then poured around the injury tube to obtain a perfect seal. After the acrylic has hardened, the scalp is stapled/sutured back. Animals are removed from the anesthesia and returned to their home cage.

Day 2: 24 hours after the previous injury cap preparation, the rats are reanesthetized with 0.5-5% isoflurane via a custom built anesthesia chamber, the animal is placed on the table and anesthesia is administered via a nose cone until catheters are placed and the animals is intubated. A catheter is placed in the right femoral artery or tail artery to monitor arterial blood pressure and blood gases. Brain temperature is indirectly measured by a thermistor placed in the left temporalis muscle and maintained at a normothermic (37° C.) level prior and subsequent to TBI. Rectal temperature is also maintained at normothermic levels. After intubation, the animal is connected to a respirator and ventilated with 0.5-5% isoflurane in a mixture of 70% nitrous oxide and 30% oxygen. 14G IV catheters are used for the ventilation tube which is modified to an appropriate length. The ventilation rate is 48 to 58 strokes per minute and the tidal volume is 2.5-3.5 and adjusted for the weight of the animal. The animal is paralyzed with rocuronium or pancuronium or vencuronium for mechanical ventilation to maintain arterial blood gases within normal limits. The fluid percussion device consists of a plexiglass cylindrical reservoir bounded at one end by a rubber-covered plexiglass piston with the opposite end fitted with a transducer housing and a central injury screw adapted for the rat’s skull. The entire system is filled with isotonic saline. The (aseptic) metal injury screw is next firmly connected to the plastic injury tube of the intubated and anesthetized rat. The injury is induced by the descent of a metal pendulum striking the piston, thereby injecting a small volume of saline epidurally into the closed cranial cavity and producing a brief displacement (18 msec) of neural tissue. The amplitude of the resulting pressure pulse is measured in atmospheres by a pressure transducer and recorded on a PowerLab chart recording system. Sham animals undergo all surgical procedures but are not subjected to the fluid percussion pulse. In the experiments, a moderate (1.8-2.2 atm) injury is studied. Animals receive Buprenorphine after the TBI. After either the TBI or sham injury, the injury cap is removed and the scalp is closed using staples. The area around the femoral artery is prepped for sterility. The sterile incision for femoral artery cannulations is stapled as well. For tail artery incisions, the tail is sutured together with sterile sutures. After 45 min-1.5 hours, the animal awakens and is moved to an individual cage supplied with food and water until termination of the study. If the animal has difficulty eating, then the animal is humanely euthanized. The rats (pre- and post-injury) in this experiment are fed per the manufacturer’s recommended daily amount of 6 pellets per day for rats. Staples or sutures are removed 10-14 days post-injury after briefly placing the animal under isoflurane anesthesia.

### Blast Injury

All animals are anesthetized and placed in an animal holding tube inserted and secured one-foot within the end of the condensing tube. The animal holding tube positions the animal with the rat’s dorsal head surface to the on-coming shock wave. Subjects are positioned 10 feet from the tube film diaphragm and receive a BOP wave in a head-on orientation. The holding tube allows for isoflurane gas to feed to the animal to induce anesthesia allowing exposures to live but anesthetized animals. BOP waves are measured and displayed for peak intensities, rise time and BOP wave durations using a Pacific Instruments 6000 DAQ with up to 32 channels, each with 250 kHz recording speed along with Dytran pressure transducers rated for 0 50 PSI measurement range and electronic conditioners interfaced with computers. An exposure consists of anesthetized animals receiving a single blast wave exposure. Investigations examine the effects of single 10-20 psi (Friedlander wave with overpressure-underpressure sequence) which have been shown to demonstrate pathological effects.

### Production of PTSD Predatory Threat

Rats are moved to special plastic cages which contain male cat urine for 10 minutes. This exposure creates a lasting PTSD phenotype in a humane fashion (See Goswami et al. Front Behav Neurosci. 2012 6:26). This cat urine exposure takes place prior to any TBI insult.

### Repeated Blast Model

A body of work has shown that repeated exposure of anesthetized rats to low level blast produces a PTSD Phenotype (See Perez-Garcia et al. Neuropharmacology. 2019 145(Pt B):220-229). In order to produce this effect rats are exposed to blast as was described earlier. This blast is repeated for three consecutive days. This PTSD model is first performed on one separate group of animals. This exposure produces both an mTBI and PTSD phenotype and therefore does not have to be combined with any other exposure.

### Outcome Measures

A variety of outcome measures are performed on all of the animals in this experiment. All outcome measures have been shown to be sensitive to changes that occur after mTBI, PTSD, and both disorders.

### Auditory Startle Response

In this outcome measure, a special Plexiglas soundproof tube attached to an accelerometer and a special auditory speaker system is used. (SR labs, San Diego CA USA; See Pooley et al. Biol Sex Differ. 2018 9(1):32). The device is calibrated at regular intervals to measure sound levels. Rats with no pre-training are placed in the tube and given 5 minutes to acclimatize with 68 dB background white noise. After five minutes the rats are exposed to a 50 ms of 110 dB tone delivered every 30 seconds for 15 minutes. Peak whole body startle response is measured every 1 ms for 100 ms after the startle exposure in an automated fashion. The average peak value per rat is normalized by body weight to obtain a value.

### Light-Dark Emergence Tasks

A light dark emergence task is performed by placing rats in a specially designed box/chamber that has a dark and lighted side separated by a tunnel. Rats naturally seek the lighted side. See Perez-Garcia et al. (2018). In this experiment rats are placed in the specially designed box for 5 minutes. The rats are placed initially in the dark side and three outcomes are measured 1) Amount of time in seconds required to reach lighted side, 2) Number of the rat entries into the lighted side, 3) Total amount of time spent in the lighted side. There are no special preparations required to perform this test and no training is required.

### Sensorimotor Testing

Spontaneous Forelimb Use: This test, described by Schallert and Lindner (Can J Psychol. 1990 44(2):276-292), assesses forelimb use during voluntary, spontaneous activity by evaluating the propensity of animals to adduct their forelimbs while rearing or standing. Animals are videotaped in a clear plastic cylinder for 5 minutes. The videotapes are scored in terms of forelimb-use asymmetry during vertical movements along the wall of the cylinder and for landings after a rear: (a) independent use of the left or right forelimb for contacting the wall of the cylinder during a full rear, to initiate a weight-shifting movement or to regain center of gravity while moving laterally in a vertical posture along the wall; Wall lands/movements and floor lands are each expressed in terms of (a) percent use of the ipsilateral (non-impaired) forelimb relative to the total number of ipsilateral and contralateral placements. During a rear, the first limb to contact the wall with clear weight support (without the other limb contacting the wall within 0.5 sec) is scored as an independent wall placement for that limb. Limb use ratio is calculated as contralateral/(ipsilateral + contralateral). This is assessed prior to brain injury as well as approximately 1 week post-trauma.

### Cognitive Testing

The analysis of cognitive function involves an assessment of spatial navigation using the water maze. Experiments that are primarily directed at assessing the activity of animals at numerous time points following TBI (such as when assessing the efficacy of therapeutic treatments designed to lessen the consequences of TBI) rely primarily on “acquisition” paradigms involving the simple place task and working memory task, in which the animals are required to learn a new platform location during each test session. This protocol does not involve pretraining or testing in the water maze prior to surgery.

General Procedures: The water maze used is a round pool (122 cm diameter; 60 cm deep) filled with water at 25° C. The maze is located in a quiet, windowless room, with a variety of distinct, extramaze cues. Four points on the rim designated as north (N), east (E), south (S), and west (W), serve as starting positions and divide the maze into four quadrants. A round platform is placed 1.5 cm beneath the surface of the water, at a location that varies according to the requirements of the task (see below). The animal’s movement is videotaped with a CCD video which records the swim path. The animal’s swim path is then analyzed with Ethovision (Noldus) software program. This program determines path length, latency to reach the platform, time spent in each quadrant of the water maze, and swim speed.

Hidden Platform Task: The platform is located in a target quadrant of the maze. Each animal receives four trials each day that may last up to 60 seconds. If the rat successfully locates the platform within the 60 seconds, it is allowed to remain for 10 seconds. Otherwise, once 60 seconds elapses, it is placed on the platform for a period of 10 seconds. Inter-trial intervals are two to four minutes, during which rats are placed under a heat lamp.

Probe Trial: This consists of removing the platform completely from the pool. The animal is released from a predetermined position and the swim pattern is recorded for 30 seconds. An animal with intact spatial memory should spend a majority of time swimming in the target quadrant that previously contained the hidden platform.

Working Memory Task: For the working memory task, the animal is given 60 seconds to find a submerged (non-cued) platform placed in a novel location within the pool. If the rat fails to find the platform within 60 seconds, the animal is placed on the platform for 10 seconds. This is considered Trial 1. Five seconds following Trial 1, a second identical trial is conducted for that same rat. Rats are placed under a heat lamp for 4 minutes between each paired trial. After running the group of rats as above, the platform is then moved to another novel location within the pool, and the paired trials are repeated. Five paired trials occur each day for 2 days.

Auditory Brainstem Response (ABR): Hearing thresholds are determined by auditory brainstem response (ABR) via subcutaneous platinum needle electrodes placed at the vertex (reference), right mastoid (negative) and the left hind limb with the animals anesthetized with ketamine (150 mg/kg) and xylazine (10 mg/kg). Digitally-generated stimuli consist of 1024 specific frequency tone bursts at between 3 and 30 kHz with a trapezoid envelop of 5 ms overall duration. The trapezoid is presented at a 3 ms plateau with 1 ms rise and fall. The stimulus is routed through a computer-controlled attenuator to an insert earphone (Etymotic Research ER-2) . The sound delivery tube of the insert earphone is positioned about 5 mm from the tympanic membrane. The output of the insert earphone is calibrated by measuring the sound pressure level at a position 4-5 mm away from the tympanic membrane. The electrical response from the recording electrode is amplified (100,000 x), filtered (100-3000 Hz) and fed to an A/D converter on a signal processing board in the computer. Eight hundred to twelve hundred samples are averaged at each level. Stimuli is presented at the rate of 16/sec and the stimulus level is varied in 10 dB descending steps, until threshold is reached, then a 5 dB ascending step to confirm. Threshold is defined as the mid-point between the lowest level at which a clear response is seen and the next lower level where no response is seen. ABR is determined as a reproducible wave II response.

### Statistics

All outcome measures yield measurable responses. The group mean response to each outcome is compared utilizing an analysis of variance with significant differences set at p less than or equal to 0.05. Comparisons are made between groups (types of treatment) in each exposure condition (e.g. NAC/PS vs. control carrier after Fluid Percussion plus PTSD stress).

1. A method for alleviating one or more symptoms of mild traumatic brain injury (mTBI), post-traumatic stress disorder (PTSD) or mTBI with PTSD, said method comprising administering to a subject suffering from mTBI, PTSD or mTBI with PTSD a psychedelic agent in combination with N-acetylcysteine (NAC).

2. The method of claim 1 wherein the one or more symptoms is selected from intrusive memories, nightmares, a sense of reliving the trauma, or psychological or physiological distress when reminded of the trauma, active avoidance of thoughts, feelings, or reminders of the trauma, inability to recall some aspect of the trauma, withdrawal from others, or emotional numbing, insomnia, irritability, difficulty concentrating, hypervigilence, or heightened startle response.

3. The method of claim 1 wherein the psychedelic agent and NAC are administered simultaneously.

4. The method of claim 3 wherein the psychedelic agent and NAC in various concentrations are formulated in a solid dosage form and administered to a patient orally.

5. The method of claim 3 wherein the psychedelic agent and NAC in various concentrations are formulated as a solution or a suspension with one or more excipients in a nonpressurized dispenser or dispensers and delivered to a patient as a nasal spray containing a metered dose of each ingredient.

6. The method of claim 3 wherein the composition is administered to prevent pathological conversion of STM to LTM and promote disengagement of pathological LTM by a chemical agonist/antagonist shock.

7. The method of claim 1 wherein the psychedelic agent is administered before NAC.

8. The method of claim 1 wherein the psychedelic agent is administered after NAC.

9. The method of claim 1 wherein the psychedelic agent is mescaline, lysergic acid diethylamide (LSD), psilocybin or a psilocybin-derived agent, or N,N-Dimethyltryptamine (DMT ) .

10. The method of 8 claim 1 wherein the psychedelic agent is psilocybin or a psilocybin-derived agent.

11. The method of claim 1 further comprising imprint pairing one or more symptoms of mTBI, PTSD or mTBI with PTSD in the subject with an odor and eliminating the subject’s ability to smell the odor.

12. A pharmaceutical composition comprising a psychedelic agent and N-acetylcysteine (NAC) and one or more excipients..

13. The pharmaceutical composition of claim 12 formulated in a solid dosage form and administered to a patient orally or in a nasal spray.

14-18. (canceled)

19. A nasal mist transducer (NMT) for administration of one or more pharmaceutically active ingredients as fine mist particles at preselected dosages and times, said NMT comprising:
a nasal funnel capable of fitting into a vestibular anatomy of a human:
a mist generator with a top and bottom which produces a fine mist at the top which is propelled toward the nasal funnel;
a syringe loading apparatus capable of holding one or more micro syringes attached at the bottom of the mist generator; and
a means for applying pressure to a plunger of a microsyringe loaded into the syringe loading apparatus.

20-23. (canceled)

24. The NMT device of claim 19 further comprising one or more preloaded micro syringes comprising selected dosages of one or more pharmaceutical ingredients positioned in the syringe loading apparatus.

25. The NMT device of claim 24 wherein the preloaded micro syringes comprising a psychedelic agent and NAC.

26-28. (canceled)

29. A method for administering one or more pharmaceutical ingredients to the circulatory system of the brain, said method comprising administering the one or more pharmaceutical ingredients via the NMT of claim 19.

30. A method for treating or alleviating symptoms associated with mTBI, PTSD or mTBI with PTSD, said method comprising administering to a subject suffering from mTBI, PTSD or mTBI with PTSD a psychedelic agent and NAC via the NMT of claim 25.
